(19) United States
(12) Patent Application Publication
(10) Pub. No.: US 2008/0241249 A1
(43) Pub. Date: Oct. 2, 2008

(54) Cyanoacrylate composite

(75) Inventors: Julian A. Quintero, Raleigh, NC (US); Jerry Y. Jonn, Shanghai (CN)

Correspondence Address: BRINKS, HOFER, GILSON & LIONE, 2801 SLATER ROAD, SUITE 120, MORRISVILLE, NC, 27560, US

(73) Assignee: Closure Medical Corporation, Raleigh, NC (US)

(21) Appl. No.: 11/731,839

(22) Filed: Mar. 30, 2007

(51) Int. Cl. A61K 9/00; A61K 31/78; A61P 17/00

(52) U.S. Cl. 424/487; 424/78.02

(57) ABSTRACT

An adhesive composite composition is provided including one or more polymerizable monomers and one or more metal stearates. The one or more polymerizable monomers may be a cyanoacrylate monomer. The adhesive composite composition may further comprise a plasticizer, an initiator, a rate modifier, a stabilizer, a colorant, a heat dissipating agent, or other additives. Methods for the application of the adhesive composite compositions to living tissue are also provided. The adhesive composite composition provides an adhesive composite material upon polymerization which is a polymer matrix entrapping the metal stearate. Polymerization of the adhesive composite composition at a site on living tissue provides an adhesive composite material which promotes microcirculation and tissue growth at the site of application of the adhesive composite composition.

### BACKGROUND

1. Field

The invention relates to adhesive composite or matrix materials, and to their use for industrial and medical applications.

2. State of the Art

Monomer and polymer adhesives are used in both industrial (including household) and medical applications. Included among these adhesives are the 1,1-disubstituted ethylene monomers and polymers, such as the α-cyanoacrylates. Since the discovery of the adhesive properties of such monomers and their resulting polymers, they have found wide use due to the speed with which they cure, the strength of the resulting bond formed, and their relative ease of use. These characteristics have made α-cyanoacrylate adhesives the primary choice for numerous applications such as bonding plastics, rubbers, glass, metals, wood, and, more recently, biological tissues.

Polymerizable 1,1-disubstituted ethylene monomers, and adhesive compositions comprising such monomers, are disclosed, for example, in U.S. Pat. No. 5,328,687 to Leung et al. Suitable methods for applying such compositions to substrates, and particularly in medical applications, are described in, for example, U.S. Pat. Nos. 5,928,611; 5,582,834; 5,575,997; and 5,624,669, all to Leung et al.

Medical applications of 1,1-disubstituted ethylene adhesive compositions include use as an alternate or an adjunct to surgical sutures and staples in wound closure as well as for covering and protecting surface wounds such as lacerations, abrasions, burns, stomatitis, sores, and other surface wounds. When an adhesive is applied, it is usually applied in its monomeric form, and the resultant polymerization gives rise to the desired adhesive bond.

A need exists for cyanoacrylate adhesive compositions with enhanced properties for use in medical applications. Such properties include suitable viscosity, biocompatibility, absorbability, flexibility and stability.

### SUMMARY

An adhesive composite material is provided comprising a polymer matrix comprising one or more biocompatible cyanoacrylate polymers and a plasticizer, and at least one metal stearate entrapped in the polymer matrix, wherein the at least one metal stearate is present in an amount of at least 10% by weight of the adhesive composite material.

The adhesive composite material may further comprise one or more of stabilizing agents, preservatives, heat dissipating agents, colorant, or combinations thereof. The metal stearate may be calcium stearate, magnesium stearate or aluminum stearate.

In an embodiment, an adhesive composite composition is provided comprising one or more biocompatible cyanoacrylate monomers, about 1 to about 20 wt. % of plasticizer, and greater than about 10 wt. % metal stearate. The metal stearate may provide enhanced viscosity and may serve to initiate polymerization of the polymerizable cyanoacrylate monomers. When used in a patient's body, the resulting polymerized adhesive composite material may comprise a porous, elastic and flexible polymer matrix.

In another embodiment, a system for treating living tissue is provided comprising a first reservoir containing a biocompatible polymerizable cyanoacrylate monomer composition, a second reservoir in a non-contacting relationship with the first reservoir containing a metal stearate, and an applicator capable of combining the biocompatible polymerizable cyanoacrylate monomer composition and metal stearate to form an adhesive composite composition and then applying the adhesive composite composition to living tissue.

In an embodiment, a method of treating living tissue is provided comprising providing a polymerizable monomer composition comprising one or more biocompatible polymerizable cyanoacrylate monomers, providing a metal stearate, mixing the polymerizable monomer composition and metal stearate to form a biocompatible adhesive composite composition comprising a suspension of the metal stearate in the polymerizable monomer composition, applying the biocompatible adhesive composite composition to living tissue in need of treatment, and allowing the monomer in the biocompatible adhesive composite composition to polymerize on the living tissue to form an adhesive composite material comprising a polymer matrix comprising metal stearate entrapped within a cyanoacrylate polymer matrix. The metal stearate is present in an amount of at least 10% by weight of the adhesive composite material.

### BRIEF DESCRIPTION OF THE DRAWINGS

FIG. 1 is a graphical representation of the modulus (PSI) of various adhesive composite materials as detailed in Example 1.

FIG. 2 is a graphical representation of the elongation at break (inches) of various adhesive composite materials as detailed in Example 1.

FIG. 3 is a graphical representation of the break stress (PSI) of various adhesive composite materials as detailed in Example 1.

### DETAILED DESCRIPTION

An adhesive composite material is provided comprising a polymer matrix comprising one or more biocompatible cyanoacrylate polymers and a plasticizer, and at least one metal stearate entrapped in the polymer matrix. The at least one metal stearate is present in an amount of at least 10% by weight of the adhesive composite material. The adhesive composite material is flexible and compliant, presenting a distinguishable form from cyanoacrylate adhesive materials previously known which do not contain a metal stearate. The adhesive composite material is a thickened, elastic, flexible, bulky, and compliant polymer. The mechanical properties of the adhesive composite material are comparable to those obtained by the use of cyanoacrylate compositions without a metal stearate, while providing advantages with regard to viscosity and flexibility.

In other embodiments, absorbable cyanoacrylate adhesive composite compositions may be prepared by combining one or more metal stearates with polymerizable cyanoacrylate monomer(s) which provide an absorbable cyanoacrylate polymer upon polymerization. The combination of one or more absorbable polymerizable cyanoacrylate monomers and one or more metal stearate results in an adhesive composite composition or material with enhanced properties, such as controlled viscosity and setting time control in the monomeric adhesive composite composition form, and flexibility, rapid partial biodegradation and pore formation once the adhesive composite composition undergoes polymerization to form a polymerized adhesive composite material which provides a polymer matrix entrapping the metal stearate.

When one or more metal stearates is combined with one or more polymerizable monomers, the metal stearate and polymerizable monomer or monomers form an adhesive composite composition. “Adhesive composite composition” as used herein refers to a combination of a metal stearate with one or more polymerizable monomers or with a composition comprising one or more polymerizable monomers. The expressions “composition comprising one or more polymerizable monomers” and “polymerizable monomer composition” are used interchangeably and are used herein to refer to a composition comprising one or more polymerizable monomers which composition may also comprise one or more additional components, such as initiator, plasticizer, inhibitor or stabilizer, preservative, rate modifier, colorant, heat dissipating agent, among others, which may be used in polymerizable monomer formulations. “Adhesive composite material” or “polymerized adhesive composite material” as used herein refers to the polymerized material or the polymer matrix formed after polymerization of the polymerizable monomer composition or the adhesive composite composition.

The metal stearate and polymerizable monomer(s) may be combined to form an adhesive composite composition by any means known to those of skill in the art, such as by bringing the components into contact, mixing, blending, distributive mixing, dispersive mixing or other means.

In forming the adhesive composite composition, when the metal stearate is combined with the polymerizable monomer or monomers, a small amount of the metal stearate becomes partially dissolved while a substantial amount or majority of the metal stearate becomes suspended in the polymerizable monomer or polymerizable monomer composition. Thus, in embodiments, the adhesive composite composition is a suspension of metal stearate in polymerizable monomer composition. “Suspension” as used herein refers to a system in which metal stearate particles or particulates are dispersed throughout a polymerizable monomer. In embodiments, the metal stearate particulates are at least microscopically visible, and may be physically and chemically separated from the polymerizable monomer composition in the adhesive composite composition.

In embodiments, the metal stearate will form a suspension when combined with a polymerizable cyanoacrylate monomer composition. Typically, a substantial portion of the metal stearate is microscopically and physically distinguishable from the polymerizable cyanoacrylate monomer composition in the adhesive composite composition thus formed. In addition, upon polymerization of the polymerizable cyanoacrylate monomer composition, a polymer matrix forms in which the metal stearate is distinguishable from the polymerized cyanoacrylate polymer matrix.

“Distinguishable” as used herein refers to the metal stearate being differentiable as a substantially separate component, e.g., a particulate component, within the suspension with the polymerizable monomer composition or, upon polymerization, within the polymer matrix. The metal stearate combined with one or more polymerizable monomers to form a composite adhesive composition provides a viscosity enhancing effect on the monomer or monomers or the monomer composition, but remains a differentiable part of the adhesive composite composition. Upon polymerization, the metal stearate in the adhesive composite material is substantially entrapped in the polymer matrix formed from the polymerizable monomer or monomers.

Without being bound to any theory, it is believed that the polymer matrix structure of the adhesive composite material, when used in the body of a patient, allows for the metal stearate to degrade or biodegrade within the polymer matrix and/or allows for the metal stearate to diffuse through and/or leach from the polymer matrix, forming a porous polymer matrix. It is further believed that the metal stearate may degrade or biodegrade or be absorbed faster than the polymer matrix can be absorbed in a patient's body, or that the metal stearate can diffuse through or leach from the polymer matrix prior to the biodegradation or absorption of the polymer matrix, forming a porous system. This porous polymer matrix may provide a structure that allows microcirculation and tissue growth through the porous polymer matrix. As used herein, “degradation” refers to any manner of the metal stearate exiting the polymer matrix which results in the formation of a porous matrix. This egress of the metal stearate from the polymer matrix is believed to form a porous matrix that promotes microcirculation and tissue growth, therefore allowing healing to take place.

The adhesive composite composition has enhanced viscosity, thus avoiding previously known problems with the use of polymerizable monomers. By way of example, one problem with using monomeric cyanoacrylate compositions in many medical applications is product run-off. This run-off may cause the material to reach unintended locations. This is a drawback in applications where precision is of importance, particularly in medical applications where the cyanoacrylate composition is applied in or on the body of a patient. The adhesive composite composition and the polymerized adhesive composite material of polymerizable cyanoacrylate monomer(s) and metal stearate provides numerous advantages, such as the elimination/reduction of run-off, precision, elasticity, material memory, flexibility, bulkiness, and overall good compliance to tissue. By way of example, an adhesive composite composition comprising at least one cyanoacrylate monomer and one or more metal stearates thus provides a thickened material with enhanced viscosity that resists run-off. The polymerized adhesive composite material provides additional advantages, including, but not limited to, microcirculation and tissue growth through the porous structure of the polymer matrix resulting from the degradation of the metal stearate from the cyanoacrylate polymer matrix.

Another problem previously known in using polymerizable cyanoacrylate monomers to form cyanoacrylate polymers was sometimes found in attaching tissue layers, such as in seroma management. Polycyanoacrylate formed from polymerizing cyanoacrylate monomer(s) may create a physical barrier that separates tissue planes that need to be in contact for appropriate healing. The adhesive composite material comprising a polymer matrix of one or more biocompatible cyanoacrylate polymers and metal stearate entrapped in the polymer matrix is believed to solve this problem at least in part through rapid partial degradation, biodegradation or diffusion of the metal stearate from the adhesive composite material when used in or on the body of a patient.

Previous attempts to solve the problems involved with seroma management included the use of surgical drains. The use of such drains increases cost, infection rates, and may cause other complications. However, when an adhesive composite composition of polymerizable cyanoacrylate monomer(s) and one or more metal stearates is used to form a cyanoacrylate polymer by polymerization of the one or more cyanoacrylate monomers, the need for surgical drains may be diminished as the dead space in the tissue may be eliminated by the adherence of the tissue planes with the polymerized cyanoacrylate composite material.

Suitable metal stearates for use in an adhesive composite composition typically are substantially insoluble in the polymerizable monomer or monomers, but may be readily combined or mixed with the polymerizable monomer or monomers. The metal stearates generally are used in the form of freely flowable powders or particulates.

Suitable metal stearates include magnesium stearate, aluminum stearate, calcium stearate, zinc stearate, or mixtures thereof. In embodiments, the metal stearate may be calcium stearate, aluminum stearate or magnesium stearate.

In embodiments, a metal stearate is selected which is non-toxic or biocompatible and may be used in medical applications. Particularly for medical uses, calcium stearate may be used as the metal stearate.

The metal stearate may function in embodiments as a viscosity enhancing agent. The increased viscosity, by way of example, provides the ability to apply the adhesive composite composition to a desired location without unwanted “run-off” from the desired location.

In embodiments, a polymerizable cyanoacrylate adhesive monomer composite composition will have an effectively enhanced viscosity if it has a viscosity of about 10 to about 10,000 centipoise, preferably about 30 to about 1,500 centipoise, as measured with a Brookfield Viscometer at 25° C. When the adhesive composite composition is to be used in medical applications internally in a patient, the enhanced viscosity preferably is about 100 to about 800 cP, as measured with a Brookfield Viscometer at 25° C. When the adhesive composite composition is to be used in medical applications externally on a patient, the enhanced viscosity preferably is about 30 to about 100 cP, as measured with a Brookfield Viscometer at 25° C.

The metal stearate in embodiments may be used in an amount above about 10% of the total adhesive composite composition and the polymerizable monomer composition may be used in an amount from about 90% to about 65%. In other embodiments, the metal stearate is used in an amount from about 10 to about 25% of the total adhesive composite composition and the polymerizable monomer composition is present in an amount from about 90% to about 75%.

Adhesive composite compositions and adhesive composite materials formed therefrom, are useful as tissue adhesives, sealants for preventing bleeding or for covering open wounds, implants for void space, and in other biomedical applications. The adhesive composite compositions and the adhesive composite materials resulting from polymerization thereof find uses in, for example, preventing body fluid leakage, sealing air leakage in the body, tissue approximation, apposing surgically incised or traumatically lacerated tissues; retarding blood flow from wounds; drug delivery; dressing burns; dressing skin or other superficial or deep tissue surface wounds (such as abrasions, chaffed or raw skin, and/or stomatitis); and aiding repair and regrowth of living tissue. Adhesive composite compositions and adhesive composite materials formed therefrom, have broad application for sealing wounds in various living tissue, internal organs and blood vessels, and can be applied, for example, on the interior or exterior of blood vessels and various organs or tissues. “Treating living tissue” as used herein refers to any of the above uses or any other use wherein the adhesive composite composition is applied on, to or into the body of a patient for either a prophylactic or therapeutic purpose. In embodiments, the treatment of living tissue will be for a medical therapeutic purpose.

Adhesive composite compositions, and polymers formed therefrom, are also useful in industrial and home applications, for example in bonding rubbers, plastics, wood, composites, fabrics, and other natural and synthetic materials.

Suitable monomers are readily polymerizable, e.g. anionically polymerizable or free radical polymerizable, or polymerizable by zwitterions or ion pairs to form polymers. Some such monomers are disclosed in, for example, U.S. Pat. No. 5,328,687 to Leung, et al., which is hereby incorporated by reference herein in its entirety. Preferred monomers include 1,1-disubstituted ethylene monomers, such as α-cyanoacrylates. Preferably, the adhesive composite compositions comprise one or more polymerizable cyanoacrylate monomers and are biocompatible. The adhesive composite compositions comprising one or more polymerizable cyanoacrylate monomers may include combinations or mixtures of cyanoacrylate monomers.

The term “biocompatible” refers to a material being suited for and meeting the requirements of a medical device, used for either long or short term implants or for non-implantable applications, such that when implanted or applied in an intended location, the material serves the intended function for the required amount of time without causing an unacceptable response. Long term implants are defined as items implanted for more than 180 days.

By way of example, useful monomers include α-cyanoacrylates of formula (I). These monomers are known in the art and have the formula

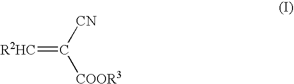

wherein R² is hydrogen and R³ is a hydrocarbyl or substituted hydrocarbyl group; a group having the formula —R⁴—O—R⁵—O—R⁶, wherein R⁴ is a 1,2-alkylene group having 2-4 carbon atoms, R⁵ is an alkylene group having 1-4 carbon atoms, and R⁶ is an alkyl group having 1-6 carbon atoms; or a group having the formula

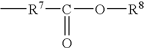

wherein R⁷ is

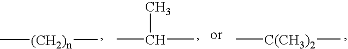

wherein n is 1-10, preferably 1-5 carbon atoms, and R⁸ is an organic moiety.

Examples of suitable hydrocarbyl and substituted hydrocarbyl groups include straight chain or branched chain alkyl groups having 1-16 carbon atoms; straight chain or branched chain C₁-C₁₆ alkyl groups substituted with an acyloxy group, a haloalkyl group, an alkoxy group, a halogen atom, a cyano group, or a haloalkyl group; straight chain or branched chain alkenyl groups having 2 to 16 carbon atoms; straight chain or branched chain alkynyl groups having 2 to 12 carbon atoms; cycloalkyl groups; aralkyl groups; alkylaryl groups; and aryl groups.

The organic moiety R⁸ may be substituted or unsubstituted and may be straight chain, branched or cyclic, saturated, unsaturated or aromatic. Examples of such organic moieties include C₁-C₈ alkyl moieties, C₂-C₈ alkenyl moieties, C₂-C₈ alkynyl moieties, C₃-C₁₂ cycloaliphatic moieties, aryl moieties such as phenyl and substituted phenyl and aralkyl moieties such as benzyl, methylbenzyl, and phenylethyl. Other organic moieties include substituted hydrocarbon moieties, such as halo (e.g., chloro-, fluoro- and bromo-substituted hydrocarbons) and oxy-substituted hydrocarbon (e.g., alkoxy substituted hydrocarbons) moieties. Preferred organic radicals are alkyl, alkenyl, and alkynyl moieties having from 1 to about 8 carbon atoms, and halo-substituted derivatives thereof. Particularly preferred are alkyl moieties of 4 to 6 carbon atoms.

In the cyanoacrylate monomer of formula (I), R³ may be an alkyl group having 1-10 carbon atoms or a group having the formula -AOR⁹, wherein A is a divalent straight or branched chain alkylene or oxyalkylene moiety having 2-8 carbon atoms, and R⁹ is a straight or branched alkyl moiety having 1-8 carbon atoms.

Examples of groups represented by the formula -AOR⁹ include 1-methoxy-2-propyl, 2-butoxy ethyl, isopropoxy ethyl, 2-methoxy ethyl, and 2-ethoxy ethyl.

The α-cyanoacrylates of formula (I) can be prepared according to methods known in the art. U.S. Pat. Nos. 2,721,858 and 3,254,111, each of which is hereby incorporated in its entirety by reference, disclose methods for preparing α-cyanoacrylates. For example, the α-cyanoacrylates can be prepared by reacting an alkyl cyanoacetate with formaldehyde in a nonaqueous organic solvent and in the presence of a basic catalyst, followed by pyrolysis of the anhydrous intermediate polymer in the presence of a polymerization inhibitor.

The α-cyanoacrylates of formula (I) wherein R³ is a group having the formula R⁴—O—R⁵—O—R⁶ can be prepared according to the method disclosed in U.S. Pat. No. 4,364,876 to Kimura et al., which is hereby incorporated in its entirety by reference. In the Kimura et al. method, the α-cyanoacrylates are prepared by producing a cyanoacetate by esterifying cyanoacetic acid with an alcohol or by transesterifying an alkyl cyanoacetate and an alcohol; condensing the cyanoacetate and formaldehyde or para-formaldehyde in the presence of a catalyst at a molar ratio of 0.5-1.5:1, preferably 0.8-1.2:1, to obtain a condensate; depolymerizing the condensation reaction mixture either directly or after removal of the condensation catalyst to yield crude cyanoacrylate; and distilling the crude cyanoacrylate to form a high purity cyanoacrylate.

The α-cyanoacrylates of formula (I) wherein R³ is a group having the formula

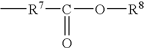

can be prepared according to the procedure described in U.S. Pat. No. 3,995,641 to Kronenthal et al., which is hereby incorporated in its entirety by reference. In the Kronenthal et al. method, such α-cyanoacrylate monomers are prepared by reacting an alkyl ester of an α-cyanoacrylic acid with a cyclic 1,3-diene to form a Diels-Alder adduct which is then subjected to alkaline hydrolysis followed by acidification to form the corresponding α-cyanoacrylic acid adduct. The α-cyanoacrylic acid adduct is preferably esterified by an alkyl bromoacetate to yield the corresponding carbalkoxymethyl α-cyanoacrylate adduct. Alternatively, the α-cyanoacrylic acid adduct may be converted to the α-cyanoacrylyl halide adduct by reaction with thionyl chloride. The α-cyanoacrylyl halide adduct is then reacted with an alkyl hydroxyacetate or a methyl substituted alkyl hydroxyacetate to yield the corresponding carbalkoxymethyl α-cyanoacrylate adduct or carbalkoxy alkyl α-cyanoacrylate adduct, respectively. The cyclic 1,3-diene blocking group is finally removed and the carbalkoxy methyl α-cyanoacrylate adduct or the carbalkoxy alkyl α-cyanoacrylate adduct is converted into the corresponding carbalkoxy alkyl α-cyanoacrylate by heating the adduct in the presence of a slight deficit of maleic anhydride.

Examples of monomers of formula (I) include cyanopentadienoates and α-cyanoacrylates of the formula:

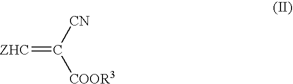

wherein Z is —CH═CH₂ and R³ is as defined above. The monomers of formula (II) wherein R³ is an alkyl group of 1-10 carbon atoms, i.e., the 2-cyanopenta-2,4-dienoic acid esters, can be prepared by reacting an appropriate 2-cyanoacetate with acrolein in the presence of a catalyst such as zinc chloride. This method of preparing 2-cyanopenta-2,4-dienoic acid esters is disclosed, for example, in U.S. Pat. No. 3,554,990, which is hereby incorporated in its entirety by reference.

Suitable α-cyanoacrylate monomers which may be used, alone or in combination, include alkyl α-cyanoacrylates such as 2-octyl cyanoacrylate; dodecyl cyanoacrylate; 2-ethylhexyl cyanoacrylate; butyl cyanoacrylate such as n-butyl cyanoacrylate; ethyl cyanoacrylate; methyl cyanoacrylate or other α-cyanoacrylate monomers such as methoxyethyl cyanoacrylate; 2-ethoxyethyl cyanoacrylate; 3-methoxybutyl cyanoacrylate; 2-butoxyethyl cyanoacrylate; 2-isopropoxyethyl cyanoacrylate; and 1-methoxy-2-propyl cyanoacrylate. In embodiments, the monomers are ethyl, n-butyl, or 2-octyl α-cyanoacrylate.

Other cyanoacrylates which may be used include alkyl ester cyanoacrylates. Besides the process detailed above, alkyl ester cyanoacrylates can also be prepared through the Knoevenagel reaction of an alkyl cyanoacetate, or an alkyl ester cyanoacetate, with paraformaldehyde. This leads to a cyanoacrylate oligomer. Subsequent thermal cracking of the oligomer results in the formation of a cyanoacrylate monomer. After further distillation, a cyanoacrylate monomer with high purity (greater than 95.0%, preferably greater than 99.0%, and more preferably greater than 99.8%), may be obtained.

Monomers prepared with low moisture content and essentially free of impurities (e.g., surgical grade) are preferred for biomedical use. Monomers utilized for industrial purposes need not be as pure.

In some embodiments, the alkyl ester cyanoacrylate monomers may have the formula:

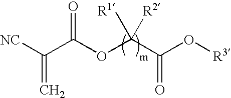

wherein R^{1′} and R^{2′} are, independently, H, a straight, branched or cyclic alkyl, or are combined together in a cyclic alkyl group, R^{3′} is a straight, branched or cyclic alkyl group, and m is 1-8. Preferably, R^{1′} is H or a C₁, C₂ or C₃ alkyl group, such as methyl or ethyl; R^{2′} is H or a C₁, C₂ or C₃ alkyl group, such as methyl or ethyl; R^{3′} is a C₁-C₁₆ alkyl group, more preferably a C₁-C₁₀ alkyl group, such as methyl, ethyl, propyl, isopropyl, butyl, isobutyl, pentyl, hexyl, heptyl, octyl, nonyl or decyl, and even more preferably a C₂, C₃ or C₄ alkyl group, and m is preferably 1-4.

Examples of the alkyl ester monomers may include, but are not limited to:

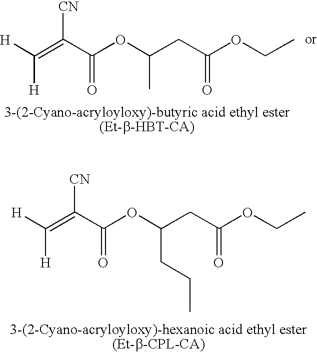

Additional examples of alkyl ester cyanoacrylates include, but are not limited to, butyl lactoyl cyanoacrylate (BLCA), butyl glycoloyl cyanoacrylate (BGCA), isopropyl glycoloyl cyanoacrylate (IPGCA), ethyl lactoyl cyanoacrylate (ELCA), and ethyl glycoloyl cyanoacrylate (EGCA) and combinations thereof. BLCA may be represented by the formula above, wherein R^{1′} is H, R^{2′} is methyl and R^{3′} is butyl. BGCA may be represented by the formula above, wherein R^{1′} is H, R^{2′} is H and R^{3′} is butyl. IPGCA may be represented by the formula above, wherein R^{1′} is H, R^{2′} is H and R^{3′} is isopropyl. ELCA may be represented by the formula above, wherein R^{1′} is H, R^{2′} is methyl and R³ is ethyl. EGCA may be represented by the formula above, wherein R^{1′} is H, R^{2′} is H and R^{3′} is ethyl.

Other examples of alkyl ester cyanoacrylates include alkyl alpha-cyanoacryloyl caprolactate and alkyl alpha-cyanoacryloyl butrylactate. Other cyanoacrylates useful in the present invention are disclosed in U.S. Pat. No. 3,995,641 to Kronenthal et al., the entire disclosure of which is hereby incorporated by reference.

Alternatively, or in addition, alkyl ether cyanoacrylate monomers may be used. Alkyl ethyl cyanoacrylates have the general formula:

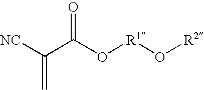

wherein R^{1″} is a straight, branched or cyclic alkyl, and R^{2″} is a straight, branched or cyclic alkyl group. Preferably, R^{1″} is a C₁, C₂ or C₃ alkyl group, such as methyl or ethyl; and R^{2″} is a C₁-C₁₆ alkyl group, more preferably a C₁-C₁₀ alkyl group, such as methyl, ethyl, propyl, isopropyl, butyl, isobutyl, pentyl, hexyl, heptyl, octyl, nonyl or decyl, and even more preferably a C₂, C₃ or C₄ alkyl group.

Examples of alkyl ether cyanoacrylates include, but are not limited to, isopropyoxy ethyl cyanoacrylate (IPECA) and methoxy butyl cyanoacrylate (MBCA) or combinations thereof. IPECA may be represented by the formula above, wherein R^{1″} is ethylene and R^{2″} is isopropyl. MBCA may be represented by the formula above, wherein R^{1″} is n-butylene and R^{2″} is methyl.

Alkyl ester cyanoacrylates and alkyl ether cyanoacrylates are particularly useful for medical applications because of their absorbability by living tissue and associated fluids. The terms “absorbable” or “absorbable adhesive” or variations thereof mean the ability of a tissue-compatible material to degrade or biodegrade at some time after implantation into products that are eliminated from the body or metabolized therein. Thus, as used herein, absorbability means that the polymerized adhesive is capable of being absorbed, either fully or partially, by tissue after application of the adhesive.

Likewise, the terms “non-absorbable” or “non-absorbable adhesive” or variations thereof mean completely or substantially incapable of being absorbed, either fully or partially, by tissue after application of the adhesive. Furthermore, relative terms such as “faster absorbing” and “slower absorbing” are used relative to two monomer species to indicate that a polymer produced from one monomer species is absorbed faster (or slower) than a polymer formed from the other monomer species.

For the purposes herein, the term “substantially absorbed” means at least 90% absorbed within about three years. Likewise, the term “substantially non-absorbed” means at most 20% absorbed within about three years. Preferably, 100% of the polymerized and applied cyanoacrylate when using these types of cyanoacrylate monomers may be absorbed in a period of less than 3 years, preferably approximately 2-24 months, more preferably 3-18 months, and most preferably 6-12 months after application of the adhesive to living tissue. The absorption time may vary depending on the particular uses and tissues involved. Thus, for example, longer absorption time may be desired where the adhesive composition is applied to hard tissues, such as bone, but a faster absorption time may be desired where the adhesive composite composition is applied to softer tissues.

The selection of monomer will affect the absorption rate of the resultant polymer, as well as the polymerization rate of the monomer. Two or more different monomers that have varied absorption and/or polymerization rates may be used in combination to give a greater degree of control over the absorption rate of the resultant polymer, as well as the polymerization rate of the monomer.

According to some embodiments, the adhesive composite composition comprises a mixture of monomer species with varying absorption rates. Where two monomer species having different absorption rates are used, it is preferred that the absorption rates be sufficiently different that a mixture of the two monomers can yield a third absorption rate that is effectively different from the absorption rates of the two monomers individually. Compositions according to these embodiments are described, for example, in U.S. patent application Ser. No. 09/919,877, filed Aug. 2, 2001, published as U.S. Patent Publication No. 2002/0037310 on Mar. 28, 2002, and U.S. Pat. No. 6,620,846, both incorporated herein by reference in their entireties.

Absorbable cyanoacrylates have broad application for closure and hemostatic sealing of wounds and the like in various living tissue, including but not limited to internal organs and blood vessels. These absorbable formulations can be applied on the interior or exterior of various organs and tissues.

Adhesive composite compositions as disclosed preferably are biocompatible and may be applied internally or externally in or on living tissue. The adhesive composite compositions are preferably sterilized for use in medical applications. More preferably, the adhesive composite compositions may be sterilized by dry heat sterilization while retaining suitability for medical applications.

For example, suitable adhesive composite compositions according to embodiments can be prepared by mixing suitable quantities of an alkyl alpha cyanoacrylate such as 2-octyl alpha-cyanoacrylate with one of butyl lactoyl cyanoacrylate (BLCA), butyl glycoloyl cyanoacrylate (BGCA), isopropyl glycoloyl cyanoacrylate (IPGCA), ethyl lactoyl cyanoacrylate (ELCA), and ethyl glycoloyl cyanoacrylate (EGCA). Such mixtures may range from ratios of about 90:10 to about 10:90 by weight, preferably about 75:25 to about 25:75 by weight such as from about 60:40 to about 40:60 by weight.

In embodiments, the metal stearate and the polymerizable monomer composition are not combined to form the adhesive composite composition until just prior to or at the time of use. Thus, the metal stearate may comprise a first component and the polymerizable monomer composition may comprise a second component in a system for treating living tissue. A two component system may be used, by way of example, where the metal stearate effectively initiates or accelerates the polymerization of the polymerizable monomer composition. Besides polymerizable monomer(s), the polymerizable monomer composition may comprise one or more additional constituents.

By way of example, stabilizing agents may be used in the polymerizable monomer composition. Suitable free radical stabilizing agents for use in polymerizable cyanoacrylate adhesive composite compositions comprising one or more polymerizable cyanoacrylate monomers include hydroquinone, hydroquinone monomethyl ether, catechol, pyrogallol, benzoquinone, 2-hydroxybenzoquinone, p-methoxy phenol, t-butyl catechol, butylated hydroxy anisole, butylated hydroxy toluene, and t-butyl hydroquinone and mixtures or combinations thereof. The free radical stabilizing agents may be used in amounts from about 5 to about 10,000 ppm. In embodiments, if hydroquinone is used, the amount may be from about 5 to about 70 ppm and may be used in conjunction with butylated hydroxy anisole in an amount of about 500 to about 10,000 ppm.

Cyanoacrylate adhesive composite compositions comprising one or more polymerizable cyanoacrylate monomers may also optionally include both at least one anionic vapor phase stabilizer and at least one anionic liquid phase stabilizer. These stabilizing agents inhibit polymerization. Examples of such anionic agents are described for example, in U.S. Pat. No. 6,620,846, incorporated herein by reference in its entirety.

The anionic vapor phase stabilizers may be selected from among known stabilizers, including, but not limited to, sulfur dioxide or hydrogen fluoride. The amount of anionic vapor phase stabilizer that is added to the monomer composition depends on the identity of the liquid phase stabilizer(s) chosen in combination with it, the monomer to be stabilized, as well as the packaging material to be used for the composition. Typically, each anionic vapor phase stabilizer is added to give a concentration of less than about 200 parts per million (ppm). In embodiments, each anionic vapor phase stabilizer is present in an amount from about 1 to about 200 ppm, preferably from about 10 to about 75 ppm, even more preferably from about 10 to about 50 ppm, and most preferably from about 10 to about 20 ppm. The amount to be used can be determined by one of ordinary skill in the art using known techniques without undue experimentation.

In embodiments, the liquid phase anionic stabilizer is a very strong acid. As used herein, a very strong acid is an acid that has an aqueous pKₐ of less than 1.0. Suitable very strong acidic stabilizing agents include, but are not limited to, very strong mineral and/or oxygenated acids. Examples of such very strong acids include, but are not limited to, sulfuric acid (pKₐ—3.0), perchloric acid (pKₐ—5), hydrochloric acid (pKₐ—7.0), hydrobromic acid (pKₐ—9), fluorosulfonic acid (pKₐ<—10), chlorosulfonic acid (pKₐ—10). In embodiments, the very strong acid liquid phase anionic stabilizer is added to give a final concentration of about 1 to about 200 ppm. The very strong acid liquid phase anionic stabilizer may be present in a concentration of from about 5 to about 80 ppm, preferably from about 10 to about 40 ppm. The amount of very strong acid liquid phase anionic stabilizer to be used can be determined by one of ordinary skill in the art without undue experimentation.

In embodiments, the very strong acid liquid phase anionic stabilizer is sulfuric acid, perchloric acid, or chlorosulfonic acid.

The polymerizable monomer composition in a cyanoacrylate adhesive composite composition may also optionally include at least one other anionic stabilizing agent that inhibits polymerization. These agents are herein referred to as secondary anionic active agents to contrast them with the strong or very strong liquid phase anionic stabilizers, which are referred to hereinbelow as “primary” anionic stabilizers. The secondary anionic active agents can be included in the compositions to adjust the cure speed of the monomer composition, for example.

The secondary anionic active agent would normally be an acid with a higher pKₐ than the primary anionic stabilizing agent and may be provided to more precisely control the cure speed and stability of the adhesive, as well as the molecular weight of the cured adhesive. Any mixture of primary anionic stabilizers and secondary active agents may be included as long as the chemistry of the composition is not compromised and the mixture does not significantly inhibit the desired polymerization rate of the monomer composition. Furthermore, the mixture should not, in medical adhesive compositions, show unacceptable levels of toxicity.

Suitable secondary anionic active agents include those having aqueous pKₐ ionization constants ranging from 2 to 8, preferably from 2 to 6, and most preferably from 2 to 5. Examples of such suitable secondary anionic stabilizing agents include, but are not limited to, organic acids, such as acetic acid (pKₐ 4.8), benzoic acid (pKₐ 4.2), chloroacetic acid (pKₐ 2.9), cyanoacetic acid, and mixtures thereof. These secondary anionic stabilizing agents may be organic acids, such as acetic acid or benzoic acid. In embodiments, the amount of acetic acid and/or benzoic acid is about 25 to about 500 ppm. The concentration of acetic acid is typically about 50 to about 400 ppm, preferably about 75 to about 300 ppm, and more preferably about 100 to about 200 ppm.

The anionic stabilizers are chosen in conjunction such that the stabilizers are compatible with the chosen polymerizable monomer composition and each other stabilizer, as well as with the packaging material and the equipment used to make and package the composition. In other words, the combination of vapor phase stabilizer(s), liquid phase stabilizer(s), and monomer should be such that a stabilized, substantially unpolymerized monomer composition is present after packaging (and sterilization, where the composition is intended for medical applications).

Other optional components may be present in the polymerizable cyanoacrylate compositions including plasticizers, colorants, preservatives, heat dissipating agents, additional stabilizing agents and the like. Typically, these components will be used in amount of up to about 25%, more preferably up to about 10%, for example, up to about 5 weight %, based on a total weight of the composite composition.

Preservatives useful in adhesive composite compositions may be anti-microbial agents. In embodiments, a preservative may be selected from among preservatives including, but not limited to, parabens and cresols. For example, suitable parabens include, but are not limited to, alkyl parabens and salts thereof, such as methylparaben, methylparaben sodium, ethylparaben, propylparaben, propylparaben sodium, butylparaben, and the like. Suitable cresols include, but are not limited to, cresol, chlorocresol, and the like. The preservative may also be selected from other known agents including, but not limited to, hydroquinone, pyrocatechol, resorcinol, 4-n-hexyl resorcinol, captan (i.e., 3a,4,7,7a-tetrahydro-2-((trichloromethyl)thio)-1H-isoindole-1,3(2H)-dione), benzoic acid, benzyl alcohol, chlorobutanol, dehydroacetic acid, o-phenylphenol, phenol, phenylethyl alcohol, potassium benzoate, potassium sorbate, sodium benzoate, sodium dehydroacetate, sodium propionate, sorbic acid, thimerosal, thymol, phenylmercuric compounds such as phenylmercuric borate, phenylmercuric nitrate and phenylmercuric acetate, formaldehyde, and formaldehyde generators such as the preservatives Germall II® and Germall 115® (imidazolidinyl urea, available from Sutton Laboratories, Charthan, N.J.). Other suitable preservatives are disclosed in U.S. Pat. No. 6,579,469, the entire disclosure of which is hereby incorporated by reference. In embodiments, mixtures of two or more preservatives may also be used.

Adhesive composite compositions may also include a heat dissipating agent. Heat dissipating agents include liquids or solids that may be soluble or insoluble in the monomer. The liquids may be volatile and may evaporate during polymerization, thereby releasing heat from the composition. Suitable heat dissipating agents may be found in U.S. Pat. No. 6,010,714 to Leung et al., the entire disclosure of which is incorporated herein.

The adhesive composite compositions may also optionally include at least one plasticizing agent that imparts flexibility to the polymer formed from the monomer. The plasticizing agent preferably contains little or no moisture and should not significantly affect the stability or polymerization of the monomer. Such plasticizers are useful in polymerized compositions to be used for closure or covering of wounds, incisions, abrasions, sores or other applications where flexibility of the adhesive is desirable. In embodiments, the polymer matrix formed includes one or more biocompatible cyanoacrylate polymers and a plasticizer.

Examples of suitable plasticizers include acetyl tributyl citrate, dimethyl sebacate, dibutyl sebacate, triethyl phosphate, tri(2-ethylhexyl)phosphate, tri(p-cresyl) phosphate, glyceryl triacetate, glyceryl tributyrate, diethyl sebacate, dioctyl adipate, isopropyl myristate, butyl stearate, lauric acid, trioctyl trimellitate, dioctyl glutarate, polydimethylsiloxane, and mixtures thereof. In embodiments, plasticizers may include tributyl citrate, acetyl tributyl citrate or dibutyl sebacate. In embodiments, suitable plasticizers include polymeric plasticizers, such as polyethylene glycol (PEG) esters and capped PEG esters or ethers, polyester glutarates and polyester adipates.

The addition of plasticizing agents in amounts ranging from about 0.5 wt. % to about 25 wt. %, or from about 1 wt. % to about 20 wt. %, or from about 5 wt. % to about 20 wt. %, provides increased elongation and toughness of the polymerized monomer over polymerized monomers not having plasticizing agents.

The polymerizable monomer composition in the adhesive composite composition may also optionally include at least one thixotropic agent. Suitable thixotropic agents are known to the skilled artisan and include, but are not limited to, silica gels such as those treated with a silyl isocyanate. Examples of suitable thixotropic agents are disclosed in, for example, U.S. Pat. No. 4,720,513, the disclosure of which is hereby incorporated in its entirety.

The polymerizable monomer composition in the adhesive composite composition may also optionally include at least one natural or synthetic rubber to impart impact resistance, which is preferable especially for industrial compositions. Suitable rubbers are known to the skilled artisan. Such rubbers include, but are not limited to, dienes, styrenes, acrylonitriles, and mixtures thereof. Examples of suitable rubbers are disclosed in, for example, U.S. Pat. Nos. 4,313,865 and 4,560,723, the disclosures of which are hereby incorporated in their entireties.

Adhesive composite compositions for medical uses may also include at least one biocompatible agent effective to reduce active formaldehyde concentration levels produced during in vivo biodegradation of the polymer (also referred to herein as “formaldehyde concentration reducing agents”). Preferably, this component is a formaldehyde scavenger compound. Examples of useful formaldehyde scavenger compounds include sulfites; bisulfites; and mixtures of sulfites and bisulfites, among others. Useful additional examples of formaldehyde scavenger compounds and methods for their implementation may be found U.S. Pat. Nos. 5,328,687, 5,514,371, 5,514,372, 5,575,997, 5,582,834 and 5,624,669, all to Leung et al., which are hereby incorporated herein by reference in their entireties. A preferred formaldehyde scavenger is sodium bisulfite.

In embodiments, the formaldehyde concentration reducing agent is added in an effective amount to the cyanoacrylate. The “effective amount” is that amount sufficient to reduce the amount of formaldehyde generated during subsequent in vivo biodegradation of the polymerized cyanoacrylate. This amount will depend on the type of active formaldehyde concentration reducing agent, and can be readily determined without undue experimentation by those skilled in the art.

The formaldehyde concentration reducing agent may be used in either free form or in microencapsulated form. When microencapsulated, the formaldehyde concentration reducing agent is released from the microcapsule continuously over a period of time during the in vivo biodegradation of the cyanoacrylate polymer.

The microencapsulated form of the formaldehyde concentration reducing agent is preferred because this embodiment prevents or substantially reduces polymerization of the cyanoacrylate monomer by the formaldehyde concentration reducing agent, which increases shelf-life and facilitates handling of the monomer composition during use. Microencapsulation techniques are disclosed in U.S. Pat. No. 6,512,023, incorporated herein by reference in its entirety.

In embodiments, the adhesive composite composition may be applied by any means known to those of skill in the art. By way of example, any suitable applicator may be used to apply the adhesive composite composition to a substrate.

Metal stearates may function as initiators which start the polymerization of the polymerizable monomer composition and/or accelerators which speed up the polymerization. In these embodiments, maintaining the metal stearate and polymerizable monomer composition separately is preferred. In another embodiments, a rate modifier may be added to the monomer composition to further control or delay polymerization for certain applications. A rate modifier may be used, for example, to slow down polymerization where the intended use requires delayed polymerization for application of the adhesive composite composition. Acidic components, such as sulfuric acid, may be useful for this purpose.

By way of example, where the polymerizable monomer or monomers are cyanoacrylate monomers, it is preferred that the cyanoacrylate monomer or monomers and the components which may be associated with the cyanoacrylate monomer(s), such as inhibitors, plasticizers, preservatives and so on, as described, are kept separate from the metal stearate until the time of use. By way of example, the polymerizable cyanoacrylate monomer or monomers and any additives such as plasticizer, inhibitor, preservative or other desired additive may form a polymerizable cyanoacrylate monomer composition which is kept separate or in a non-contacting relationship from the metal stearate until the time of use. At or just prior to the time the adhesive composite composition is to be used, the separate polymerizable monomer composition and the metal stearate component are combined to form the adhesive composite composition.

Applicators which enable the separation of components until use and enable combination of two-component systems are well-known in the art. By way of example, the Applicator for CoSeal Sealant, distributed by Angiotech Pharmaceutical, may be used. By way of example, a two-part syringe system may be used wherein the metal stearate is in one part and the polymerizable monomer composition is in another part. The components may be pushed together, combining at the time of use to form the adhesive composite composition which is dispersed for the desired application. Such a syringe system, for example, may utilize a T-shape or straight configuration. Other two component systems are shown, for example, in U.S. Pat. Nos. 5,814,022 and 5,935,437.

In embodiments where either the metal stearate does or does not have activity as an initiator and/or an accelerator, suitable initiators and/or accelerators may be used with the polymerizable monomers. Such suitable initiators are known in the art and are described, for example, in U.S. Pat. Nos. 5,928,611 and 6,620,846, both incorporated herein by reference in their entireties, and U.S. Patent Application No. 2002/0037310, also incorporated herein by reference in its entirety. Quaternary ammonium chloride and bromide salts useful as polymerization initiators are particularly suitable. By way of example, quaternary ammonium salts such as domiphen bromide, butyrylcholine chloride, benzalkonium bromide, acetyl choline chloride, among others, may be used.

Benzalkonium or benzyltrialkyl ammonium halides such as benzyltrialkyl ammonium chloride may be used. When used, the benzalkonium halide may be benzalkonium halide in its unpurified state, which comprises a mixture of varying chain-length compounds, or it can be any suitable purified compound including those having a chain length of from about 12 to about 18 carbon atoms, including but not limited to C12, C13, C14, C15, C16, C17, and C18 compounds. By way of example, the initiator may be a quaternary ammonium chloride salt such as benzyltrialkyl ammonium chloride (BTAC).

Other initiators or accelerators may also be selected by one of ordinary skill in the art without undue experimentation. Such suitable initiators or accelerators may include, but are not limited to, detergent compositions; surfactants: e.g., nonionic surfactants such as polysorbate 20 (e.g., Tween 20™ from ICI Americas), polysorbate 80 (e.g., Tween 80™ from ICI Americas) and poloxamers, cationic surfactants such as tetrabutylammonium bromide, anionic surfactants such as sodium tetradecyl sulfate, and amphoteric or zwitterionic surfactants such as dodecyldimethyl(3-sulfopropyl)ammonium hydroxide, inner salt; amines, imines and amides, such as imidazole, arginine and povidine; phosphines, phosphites and phosphonium salts, such as triphenylphosphine and triethyl phosphite; alcohols such as ethylene glycol, methyl gallate; tannins; inorganic bases and salts, such as sodium bisulfite, calcium sulfate and sodium silicate; sulfur compounds such as thiourea and polysulfides; polymeric cyclic ethers such as monensin, nonactin, crown ethers, calixarenes and polymeric-epoxides; cyclic and acyclic carbonates, such as diethyl carbonate; phase transfer catalysts such as Aliquat 336; organometallics such as cobalt naphthenate and manganese acetylacetonate; and radical initiators or accelerators and radicals, such as di-t-butyl peroxide and azobisisobutyronitrile.

In embodiments, mixtures of two or more, such as three, four, or more, initiators or accelerators can be used. A combination of multiple initiators or accelerators may be beneficial, for example, to tailor the initiator of the polymerizable monomer species. For example, where a blend of monomers is used, a blend of initiators may provide superior results to a single initiator. For example, the blend of initiators can provide one initiator that preferentially initiates one monomer, and a second initiator that preferentially initiates the other monomer, or can provide initiation rates to help ensure that both monomer species are initiated at equivalent, or desired non-equivalent, rates. In this manner, a blend of initiators can help minimize the amount of initiator necessary. Furthermore, a blend of initiators may enhance the polymerization reaction kinetics.

In embodiments, an applicator may include an applicator body, which is formed generally in the shape of a tube having a closed end, an open end, and a hollow interior lumen, which holds a crushable or frangible ampoule. An applicator may include an ampoule for the polymerizable monomer composition and an ampoule for the viscosity enhancing agent. The applicator and its related packaging may be designed as a single-use applicator or as a multi-use applicator. Suitable multi-use applicators are disclosed, for example, in U.S. Pat. No. 6,802,416 issued Oct. 12, 2004, the entire disclosure of which is incorporated herein by reference.

In embodiments, the applicator may comprise elements other than an applicator body and an ampoule. For example, an applicator tip may be provided on the open end of the applicator. The applicator tip material may be porous, absorbent, or adsorbent in nature to enhance and facilitate application of the composition within the ampoule. Suitable designs for applicators and applicator tips that may be used according to the present invention are disclosed in, for example, U.S. Pat. Nos. 5,928,611, 6,428,233, 6,425,704, 6,455,064, and 6,372,313, the entire disclosures of which are incorporated herein by reference.

In embodiments, an applicator may contain an initiator or accelerator on a surface portion of the applicator or applicator tip, or on the entire surface of the applicator tip, including the interior and the exterior of the tip. When the initiator or accelerator is contained in or on an applicator tip, the initiator or accelerator may be applied to the surface of the applicator tip or may be impregnated or incorporated into the matrix or internal portions of the applicator tip, depending on the use. Additionally, the initiator or accelerator, when used, may be incorporated into the applicator tip, for example, during the fabrication of the tip.

In other embodiments, an initiator may be coated on an interior surface of the applicator body and/or on an exterior surface of an ampoule or other container disposed within the applicator body, may be placed in the applicator body in the form of a second frangible vial or ampoule and/or may be otherwise contained within the applicator body, so long as a non-contacting relationship between the polymerizable monomer composition and the initiator is maintained until use of the adhesive.

In embodiments, a system for treating living tissue is provided with a first reservoir containing a biocompatible polymerizable monomer composition, a second reservoir in non-contacting relationship with the first reservoir containing a metal stearate, and an applicator. The metal stearate preferably comprises calcium stearate. The biocompatible polymerizable monomer composition preferably comprises one or more cyanoacrylate monomers. The applicator is capable of combining the biocompatible polymerizable monomer composition and metal stearate to form an adhesive composite composition and applying the adhesive composite composition to living tissue. The applicator may further contain an initiator or accelerator in or on the applicator tip or in or on the interior of the applicator. The system may be present in a kit combination.

When the polymerizable monomer composition and the metal stearate are combined, the two components typically are sufficiently combined to provide a suspension of the metal stearate in the polymerizable monomer composition wherein the resulting adhesive composite composition has enhanced viscosity. Therefore, combining the two components may include mixing the two components sufficiently to provide the desired suspension of metal stearate in polymerizable monomer composition such that the adhesive composite composition has a desired viscosity for an intended use.

In embodiments, the polymerizable cyanoacrylate monomer composition is combined with the metal stearate to form an adhesive composite composition which is a suspension of metal stearate in polymerizable monomer just prior to or at the time the adhesive composite composition is to be applied to the intended site for its intended purpose. The suspension of metal stearate in polymerizable monomer composition provides enhanced viscosity for application, such as to a tissue area in need of treatment, such that the adhesive composite composition does not exhibit run-off and can be substantially precisely placed in or on a patient's body as desired. In addition, the metal stearate may function as an initiator, to initiate polymerization, or as an accelerator, to accelerate polymerization. Thus, the polymerizable monomer may be efficiently polymerized in sufficient time for medical uses without the addition or use of a separate polymerization initiator or accelerator.

In embodiments, the metal stearate may be placed in an applicator body in one container while the polymerizable cyanoacrylate composition is stored in another container within the applicator body, so long as a non-contacting relationship between the polymerizable monomer composition and the metal stearate is maintained until use of the adhesive composite composition.

Having generally described embodiments of adhesive composite compositions and adhesive composite materials, a further understanding can be obtained by reference to certain specific examples which are provided herein for purposes of illustration only and are not intended to be limiting unless otherwise specified.

### EXAMPLES

### Example 1

An adhesive composition material according to an embodiment was tested for mechanical properties as compared to cyanoacrylate compositions comprising cyanoacrylate and plasticizer, and cyanoacrylate alone. The experiment characterized cyanoacrylate/metal stearate composite materials regarding modulus (flexibility), elongation at break, and break stress as compared to 100% cyanoacrylate and plasticized cyanoacrylate polymers.

Nine cyanoacrylate formulations were prepared with target compositions according to the following Table 1:

TABLE 1

| | | Dibutyl | Calcium | Magnesium | |
|---|---|---|---|---|---|
| Formula- | Cyanoacrylate | Sebacate | Stearate | Stearate | Stearate |
| tion | (CA)* | (DBS) | (CaSt) | (MaSt) | (St) |
| # | (g) | (g) | (g) | (g) | % |
| 1 | 1.65 | 0.45 | 0 | 0.9 | 30 |
| 2 | 1.65 | 0.45 | 0.9 | 0 | 30 |
| 3 | 1.95 | 0.45 | 0 | 0.6 | 20 |
| 4 | 1.95 | 0.45 | 0.6 | 0 | 20 |
| 5** | 1.95 | 0.45 | 0.6 | 0 | 20 |
| 6 | 2.25 | 0.45 | 0 | 0.3 | 10 |
| 7 | 2.25 | 0.45 | 0.3 | 0 | 10 |
| 8 | 8.5 | 1.5 | 0 | 0 | 0 |
| 9 | 10 | 0 | 0 | 0 | 0 |

*Blend of 25% Butyl lactoyl cyanoacrylate and 75% 2-Octylcyanoacrylate
**2-Octyl cyanoacrylate

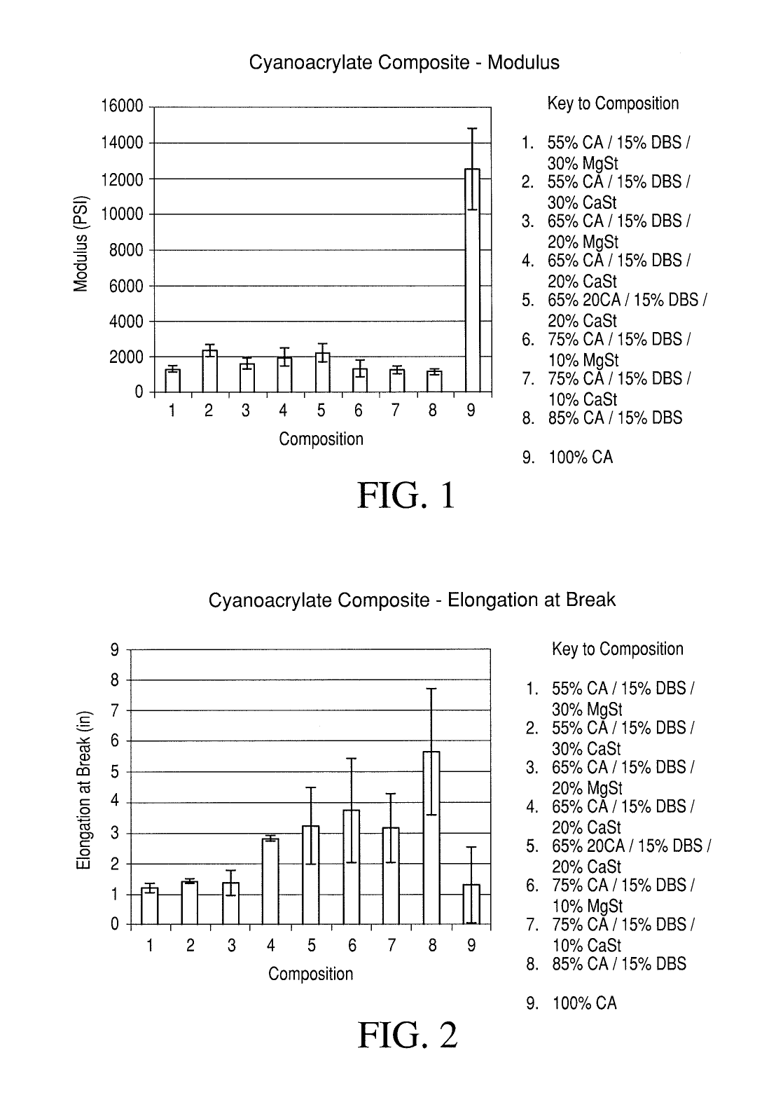

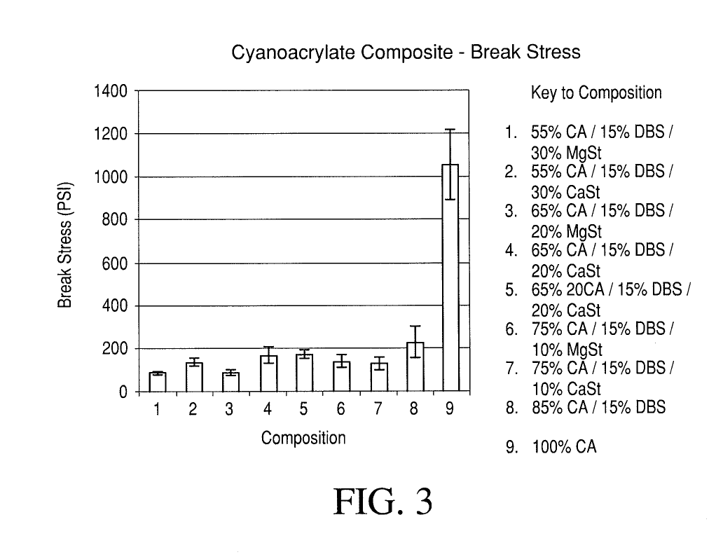

Each formulation was allowed to polymerize sandwiched between two HDPE (high density polyethylene) surfaces with a target spacing of 0.027 inches. Once polymerized, each film was cut to a width of 0.4 inches and a length of at least 2 inches, and placed in the clamps of a Mechanical Testing System (Sintech 2/G MTS, MTS Systems Corporation). The testing parameters were set as follows: break sensitivity=75%, break threshold=0.500 lbf, data acq. rate=10.0 Hz, initial speed=3.0 in/min, and secondary speed=3.0 in/min. All samples were tested for modulus, elongation at break and break stress. The results are graphically presented in FIGS. 1-3.

The polymers of cyanoacrylate formulations containing calcium or magnesium stearates, and DBS have lower moduli when compared to 100% CA (formulation # 9). This flexibility or lack of brittleness makes these types of composite materials suitable for soft tissue repair in certain surgical applications. The test results indicate that the mechanical properties are not adversely affected by the use of metal stearates as compared to cyanoacrylate compositions not containing metal stearate.

### Example 2

An adhesive composite material comprising a metal stearate in a polymer matrix of cyanoacrylate polymer provides advantages with regard to flexibility not found with materials without the metal stearate. Magnified photographs (10×) were viewed of the differing structures of an adhesive composite material according to an embodiment as disclosed compared to polymerized cyanoacrylate not containing a metal stearate.

The experiment was conducted to capture the particle dispersion of calcium stearate in cyanoacrylate polymer via photography.

A mixture of 1.4997 g of cyanoacrylate (blend of 25% butyl lactoyl cyanoacrylate and 75% 2-octylcyanoacrylate, D&C violet # 2 at 0.1%, and dibutyl sebacate at ˜20%, all by weight) and 0.9320 g of calcium stearate were vortexed in a 20 mL scintillation vial. The subsequent mixture was drawn and expressed back into the vial 10 times using a 3 cc tuberculin syringe. This produced a composite material with a composition of about 49.4% cyanoacrylate, 12.3% dibutyl sebacate, and 38.3% calcium stearate. Upon completion of mixing, the mixture was allowed to polymerize for 5 minutes inside the barrel of the syringe. Once polymerized, a cross section of about 1 mm in thickness was obtained and photographed at 10× magnification.

Two additional photographs of the cross sections of polymerized cyanoacrylate and polymerized cyanoacrylate (containing ˜20% dibutyl sebacate), both without calcium stearate, were obtained as references. One was a cross section of polymerized cyanoacrylate, and the other was a cross section of polymerized cyanoacrylate containing dibutyl sebacate. A comparison of the photographs shows that the dispersion of calcium stearate in the polymer matrix scatters light when compared to the cyanoacrylate compositions without calcium stearate. The surface of the calcium stearate cross section is rough and porous-like in nature when compared to sections of the cyanoacrylate compositions alone.

While the invention has been described with reference to preferred embodiments, the invention is not limited to the specific examples given, and other embodiments and modifications can be made by those skilled in the art without departing from the spirit and scope of the invention.

1. An adhesive composite material comprising:
a) a polymer matrix comprising one or more biocompatible cyanoacrylate polymers and a plasticizer, and
b) at least one metal stearate entrapped in the polymer matrix, wherein the at least one metal stearate is present in an amount of at least 10% by weight of the adhesive composite material.

2. The adhesive composite material of claim 1 further comprising one or more of a stabilizing agent, a preservative, a heat dissipating agent, a colorant, or a combination thereof.

3. The adhesive composite material of claim 1 wherein the one or more biocompatible cyanoacrylate polymers are formed from one or more polymerizable cyanoacrylate monomers.

4. The adhesive composite material of claim 3 wherein the one or more polymerizable cyanoacrylate monomers is 2-octylcyanoacrylate, butyl lactoyl cyanoacrylate or a mixture thereof.

5. The adhesive composite material of claim 1 wherein the metal stearate is calcium stearate, magnesium stearate, or aluminum stearate.

6. The adhesive composite material of claim 3 wherein the one or more polymerizable cyanoacrylate monomers is an alkyl α-cyanoacrylate monomer, an alkyl ester cyanoacrylate monomer or a mixture thereof.

7. The adhesive composite material of claim 6 wherein the one or more polymerizable cyanoacrylate monomers is octyl cyanoacrylate; dodecyl cyanoacrylate; 2-ethylhexyl cyanoacrylate; methoxyethyl cyanoacrylate; 2-ethoxyethyl cyanoacrylate; butyl cyanoacrylate; ethyl cyanoacrylate; methyl cyanoacrylate; 3-methoxybutyl cyanoacrylate; 2-butoxyethyl cyanoacrylate; 2-isopropoxyethyl cyanoacrylate; 1-methoxy-2-propyl cyanoacrylate; butyl lactoyl cyanoacrylate; butyl glycoloyl cyanoacrylate; isopropyl glycoloyl cyanoacrylate; ethyl lactoyl cyanoacrylate; ethyl glycoloyl cyanoacrylate; isopropyoxy ethyl cyanoacrylate; methoxy butyl cyanoacrylate; or mixtures thereof.

8. The adhesive composite material of claim 1 wherein the plasticizer is present in an amount of about 5 to about 20 wt. %.

9. The adhesive composite material of claim 5 wherein the adhesive composite material is sterile.

10. A system for treating living tissue comprising:
a first reservoir containing a biocompatible polymerizable cyanoacrylate monomer composition,
a second reservoir in a non-contacting relationship with the first reservoir containing a metal stearate, and
an applicator capable of combining the biocompatible polymerizable cyanoacrylate monomer composition and metal stearate to form an adhesive composite composition and then applying the adhesive composite composition to living tissue.

11. The system of claim 10 wherein the biocompatible polymerizable cyanoacrylate monomer composition comprises an alkyl α-cyanoacrylate monomer, an alkyl ester cyanoacrylate monomer or a mixture thereof.

12. The system of claim 11 wherein metal stearate is calcium stearate, magnesium stearate or aluminum stearate.

13. A method of treating living tissue, comprising:
providing a polymerizable monomer composition comprising one or more biocompatible polymerizable cyanoacrylate monomers,
providing a metal stearate,
mixing the polymerizable monomer composition and metal stearate to form a biocompatible adhesive composite composition comprising a suspension of the metal stearate in the polymerizable monomer composition,
applying the biocompatible adhesive composite composition to living tissue in need of treatment, and
allowing the monomer in the biocompatible adhesive composite composition to polymerize on the living tissue to form an adhesive composite material comprising a polymer matrix comprising metal stearate entrapped within a cyanoacrylate polymer matrix
wherein the metal stearate is present in an amount of at least 10% by weight of the adhesive composite material.

14. The method of claim 13 wherein the metal stearate degrades faster than the cyanoacrylate polymer matrix is absorbed into the living tissue, or the metal stearate diffuses through the cyanoacrylate polymer matrix, forming a porous cyanoacrylate polymer matrix.

15. The method of claim 13 wherein the biocompatible adhesive composite composition is sterilized prior to being applied to living tissue.

16. The method of claim 13 wherein the polymerizable monomer composition comprises an alkyl α-cyanoacrylate monomer, an alkyl ester cyanoacrylate monomer or a mixture thereof; a plasticizer; and a stabilizer.

17. An adhesive composite composition comprising one or more biocompatible cyanoacrylate monomers, about 5 to about 20 wt. % of plasticizer, and greater than about 10 wt. % metal stearate.

18. The adhesive composite composition of claim 17 wherein the metal stearate is calcium stearate, magnesium stearate, or aluminum stearate.

19. The adhesive composite composition of claim 18 wherein the one or more biocompatible cyanoacrylate monomer is octyl cyanoacrylate; dodecyl cyanoacrylate; 2-ethylhexyl cyanoacrylate; methoxyethyl cyanoacrylate; 2-ethoxyethyl cyanoacrylate; butyl cyanoacrylate; ethyl cyanoacrylate; methyl cyanoacrylate; 3-methoxybutyl cyanoacrylate; 2-butoxyethyl cyanoacrylate; 2-isopropoxyethyl cyanoacrylate; 1-methoxy-2-propyl cyanoacrylate; butyl lactoyl cyanoacrylate; butyl glycoloyl cyanoacrylate; isopropyl glycoloyl cyanoacrylate; ethyl lactoyl cyanoacrylate; ethyl glycoloyl cyanoacrylate; isopropyoxy ethyl cyanoacrylate; methoxy butyl cyanoacrylate; or mixtures thereof.

20. The adhesive composite composition of claim 19 wherein the plasticizer is dibutyl sebacate.
